(19) United States
(12) Patent Application Publication
(10) Pub. No.: US 2007/0098671 A1
(43) Pub. Date: May 3, 2007

(54) Composition and method of treatment for irritated skin

(76) Inventor: Gregory D. Martin, Dover, DE (US)

Correspondence Address: ADVANTIA LAW GROUP, 9035 SOUTH 1300 EAST, SUITE 200, SANDY, UT, 84094, US

(21) Appl. No.: 11/265,827

(22) Filed: Nov. 2, 2005

(51) Int. Cl. A61K 8/97; A61K 35/64; A61K 36/886

(52) U.S. Cl. 424/74; 424/539; 424/725; 424/744

(57) ABSTRACT

A composition for application to irritated skin including honey, aloe, and a derivative of the mangosteen plant. The composition may also include a thickening agent. Also disclosed is a method of treating skin including the step of applying the composition to the skin.

### BACKGROUND OF THE INVENTION

1. Field of the Invention

The present invention relates to compositions and methods of treatment for irritated skin, specifically compositions including honey and aloe.

2. Description of the Related Art

The use of a substance mixture comprising honey and olive oil for preparing a medicament for topical application is suggested in U.S. Pat. No. 6,482,442. This patent suggests that for treatment of patients afflicted with hemorrhoids, the application of an ointment with the following composition: honey (55%), wherein 30% of honey from blossoms, 30% of acacia (robinia) honey and 40% of wild honey are contained, olive oil (8.5%), propolis (5% of a 40% tincture), beeswax (18%), camomile tincture, sage tincture, lavender, thyme, aloe vera, wheatgerm oil.

U.S. Pat. No. 4,761,285 discloses a composition comprising Leptandra Culver's root, chick-peas and grape seeds. The composition can be enhanced with honey, cinnamon and/or oil. The composition is useful for the relief and treatment of hemorrhoids and hemorrhoidal symptoms. The composition can also be used as an herbal food supplement for dietary and nutritional purposes.

A patent that discloses the use of honey wax for treatment is U.S. Pat. No. 5,997,876. This invention refers to the sphere of medicine, in particular to pharmaceutics and concerns the preparations for burn treatment. The technical result of the present invention is in widening the assortment of medicinal preparations for treatment of burns and wounds, in increasing treatment effect. The ointment contains the following components: celandine, plantain, matricary, yarrow, calendula, St. John's wort, gum-tree, olive oil, and yellow wax of honey.

Honey used in wound dressing is disclosed in U.S. Patent Application Publication No. 2004/0054313. This invention is directed to the use of honey in medical dressings. Preferred embodiments modify honey with a viscosity increasing agent, resulting in a range of possible compositions including ointments and salves, self-adhesive gels such as for use on mouth ulcers and pustules, and pliable or flexible sheets which can be used as a wound covering. Preferred viscosity increasing agents include both particulate and continuous gels, respective examples of each including agars and alginates. Selected honeys preferably, but not necessarily, exhibit antibacterial properties other than what is merely conferred by osmolarity and sugar concentration effects.

Further, P. C. Molan of the Honey Research Unit, University of Waikato, Hamilton, New Zealand discloses the antibacterial properties of honey in an article entitled “The Potential of Honey to Promote Oral Wellness” in General Dentistry, 2001. Honey has been used as a medicine throughout the ages, and in more recent times has been “rediscovered” by the medical profession for treatment of burns, infected wounds and skin ulcers. The large volume of literature reporting its effectiveness indicates that honey has therapeutic features that indicate it has potential for the treatment of periodontal disease, mouth ulcers and other problems of oral health. Honey has a potent broad-spectrum antibacterial activity that rapidly clears infection from wounds when applied topically, which may make it suitable for “anti-infective” treatment of periodontal disease as well as for clearing infection in mouth ulcers and wounds from oral surgery.

Some of the disadvantages to the currently available compositions are that certain ingredients may have adverse effects to the user. Further, there is a movement to increase the availability of medicaments that include natural ingredients. Many of the currently available compositions include ingredients that are not all natural.

What is needed is a method and/or composition that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available compositions. According to one embodiment, the present invention has been developed to provide a composition for application to irritated skin, including honey, aloe, and a derivative of a mangosteen plant.

The honey may be creamed. The composition may also include a thickening agent. The thickening agent may be an alginate-based material. The thickening agent may be a polyvalent cation. The aloe may be substantially devoid of water. The composition may be from about 50 weight percent, to about 75 weight percent honey, from about 5 weight percent to about 20 weight percent aloe, and from about 1 weight percent to about 10 weight percent derivative of the mangosteen plant. The composition may be about 70 weight percent honey, about 10 weight percent aloe, and about 5 weight percent derivative of the mangosteen plant, and further comprising about 5 weight percent bees wax, about 5 weight percent propolis, and about 5 weight percent olive oil.

In another embodiment, the present invention includes a method of treating irritated skin, including: applying a composition to irritated skin, said composition including honey, aloe, and a derivative of a mangosteen plant.

The honey may be creamed. The aloe may be substantially devoid of water. The composition of the method may further include a thickening agent. The thickening agent may be an alginate-based material. The thickening agent may include a polyvalent cation. The method may further include the step of applying the composition to a device. The irritated skin may include one of the group consisting of: sunburns, chemical burns, rubbing burns, radiation burns, abrasions, cuts, episiotomies, irritations, allergic reactions, sore breasts, jock itch, cold sores, hemorrhoids, problems of portal circulation, anal fistulas, inflammation of the anus, arteriosklerosis, purulent wounds and injuries, traumatic erysipelas, duodenal ulcer, migrating cheilitis, loss of hair, dandruff, alopecia, psoriasis, seborrheic dermatitis, athletes foot, external viral, fungal, or bacterial infections, eczema, rashes, ulcers, varicose veins, dermatomycoses, acne, pustules, burns, recovery from skin grafts, exudating wounds, chapped skin, hickeys, herpes sores, bruises, insect bites, abrasions from poison ivy, animal bites, puncture wounds and combinations thereof. The composition may include from about 50 weight percent, to about 75 weight percent honey, from about 5 weight percent to about 20 weight percent aloe, and from about 1 weight percent to about 10 weight percent derivative of the mangosteen plant. The composition may include about 70weight percent honey, about 10-weight percent aloe, about 5-weight percent derivative of the mangosteen plant, about 5 weight percent bees wax, about 5-weight percent propolis, and about 5 weight percent olive oil.

According to yet another embodiment, the present invention includes a composition for application to hemorrhoids including, of honey, aloe, and a derivative of a mangosteen plant. The composition may be substantially devoid of corticoids, fragrances, and colors.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

As used herein, “comprising,” “including,” “containing,” “is,” “are,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

Hemorrhoids may be described as swollen veins in the lower portion of the rectum or anus. Hemorrhoids may be internal or external of the anus. Hemorrhoids may be treated using an ointment containing corticoids. Corticoids include steroids and steroid hormones. These steroids may be produced by the adrenal cortex or sytnthesized. Typically corticoids are administered to reduce swelling, and have the effect of reducing the body's immune response. There may be a large number of other side effects and risks that may accompany the use of corticoids. In one embodiment, the composition of the present invention substantially lacks corticoids other than those that may exist in the aloe, honey, thickening agents, or derivatives of the mangosteen plant that may be added to the composition.

The composition for application to irritated skin of the present invention includes honey, aloe, and thickening agent. The composition may be used to treat irritated skin. In one embodiment, the composition of the present invention is used to treat hemorrhoids.

Throughout this disclosure, unless stated otherwise, honey may include any constituents that are typically found in honey as produced by bees. For example, honey may include beeswax, pollen, crystallized honey, and so forth. Although well known as a food, honey may also be used as a medicine. Honey may be effective for treating infected wounds, skin ulcers, burned skin, and so forth. There is a large variety of different types of honey which, depending on their ingredients, have different properties as regards their application in the medical field. The main component of honey is invert sugar, further sugars are cane sugar, maltose, and, depending on the plant species visited, more rarely occurring sugars originating from the latter. Besides, sugar, honey contains enzymes, such as invertase, diastases, catalase, amylase, phosphatase, glucose oxidase, which, with the cooperation of the oxygen of air, convert dextrose into gluconic acid and hydrogen peroxide. The latter provides oxygen in an extremely reactive form which in turn is an excellent germ killer and preservative. One of the most important components of honey is pollen. Further found in honey may be the organic acids, malic acid, succinic acid, gluconic acid, acidic acid, formic acid, of the inorganic acids phosphoric acid and hydrochloric acid are present. Honey furthermore may contain mineral substances in a portion of up to 3%, among them Fe, Cu, P, S, K, Na, Mg, Ca, Si, Mn, Cl, Zn and, in small amounts, also vitamins (B1, B2, B6, pantothenic acid, nicotinic acid, H, folic acid and little vitamin C) and, in very small amounts, nearly all the amino acids. Furthermore, hormones, acetyl choline, which is involved in the conduction of nerve impulses, inhibines (bactericides) and vegetable dyes, such as flavones or carotines, and aromatic substances (alcohols, aldehydes, ketones and essential oils) have been found in honey.

There are many different types of honey available, and any kind of honey may be used in the various embodiments of the present invention. Honeydew honeys are rich in resins and essential oils. Also available are honeys of mixed blossoms, linden honey, melissa honey, chestnut honey, clover honey, and so forth. Honeys may have a variance of as much as a hundred-fold in their antibacterial potency.

The honey used in the present invention may be thickened. In one embodiment, the honey may be thickened by addition of a thickener, as discussed below, at any point in the production of the composition. In another embodiment, the honey may be creamed. Creaming may consist of controlling the crystallization of the sugar in honey. The crystallization may be controlled such that the crystals remain small. The creaming may be performed by any means known in the art such as mixing during crystallization.

The composition of the present invention may include a therapeutic amount of honey. The composition of the present invention includes at least about 25 weight percent, or at least about 50 weight percent, or at least about 70 weight percent; and less than about 95 weight percent, or less than about 85 weight percent, or less than about 80 weight percent honey.

The present invention may also include aloe. Aloe includes the juice of the leaves of any of the more than 240 species of aloe, or combinations thereof. Aloe with particular nutritional value comes from the Aloe Barbadensis. The Aloe Barbadensis with the most recognized nutritional value is the Aloe barbadensis Miller. Aloe has been used in topical and internal uses. It is believed, but not meant to be limiting, that the combination of aloe with honey has a synergistic effect.

Aloe typically includes at least two biologically helpful constituents. The first is B-sitosterol, an anti-inflammatory. The second is salicylic acid, which may be used as an antiseptic, and is sometimes used as a food preservative. When salicylic acid is reacted with acetic acid, the product is acetylsalicylic acid, commonly known as aspirin. Aloe is often taken orally for relief of common gastrointestinal ailments. For example, aloe may be taken orally to stabilize digestion, absorption and elimination, and regulate stomach pH levels. Aloe may clean and regulate the functions of the small and large intestines. It may normalize stool and remove hemorrhoids when taken orally.

In one embodiment, the aloe present in the composition includes powdered aloe. The aloe may be powdered by drying of the aloe. It is believed, but not meant to be limiting, that by using powdered aloe instead of liquid aloe, less or no water (which may have a detrimental effect on the honey of the composition) is then added to the composition through the addition of the aloe. Commercial sources of the dry powdered aloe of the present invention are available, for example, from Aloe Laboratories, Harlingen, Tex., USA and NHK Laboratories, Santa Fe Springs, Calif.

The composition of the present invention may include a therapeutic amount of aloe. The composition of the present invention includes at least about 1 weight percent, or at least about 5 weight percent, or at least about 8 weight percent; and less than about 50 weight percent, or less than about 25 weight percent, or less than about 12 weight percent aloe.

The composition of the present invention may also include other skin-healing, skin-soothing, and skin-moisturizing compounds. Some non-limiting examples of these compounds include: calamine lotion, zinc oxide ointment, anhydrous lanolin, petroleum jelly, glycerine, and so forth.

The composition of the present invention also may include a thickening agent. Although in some embodiments of the present invention no thickening agent is present, in some other embodiments, there may be a thickening agent. Of the available thickening agents, preferred are gelling agents. Available gelling agents include various gums and polysaccharides, alginates, and both synthetic and natural polymeric compounds. Some useful prior art references for gelling agents used in the food or medical professions include U.S. Pat. No. 4,948,575, U.S. Pat. No. 5,674,524, U.S. Pat. No. 5,197,945, U.S. Pat. No. 5,735,812. U.S. Pat. No. 5,238,685, U.S. Pat. No. 5,470,576, U.S. Pat. No. 5,738,860, U.S. Pat. No. 5,336,501, U.S. Pat. No. 5,482,932, and U.S. Patent Application Publication No. 2004/0054313. Reference is made to these documents as a background to various available viscosity-increasing agents which may find use in the present invention. In one embodiment alginate salts are used as the thickening agent. The thickening properties of alginates are often increased when used in connection with polyvalent cations. Polyvalent cations may include any known in the art, including calcium salts such as calcium chloride.

Other useful gelling agents include hydrocolloids and hydrogels. Compositions using only hydrocolloids or hydrogels as thickening agents exhibit different properties than compositions using other gelling agents. For example, compositions using only hydrocolloids or hydrogels as thickening agents tend to absorb less water than those using other gelling agents. The composition can, therefore, be engineered to be placed on areas of skin that excrete more or less moisture by the choice of the thickening agent. Further, combinations of thickening agents may be used such that the resulting composition exhibits the desired properties for the intended use.

The composition includes sufficient thickening agent to reach a predetermined thickness of the composition. In one embodiment, the composition includes enough thickening agent to increase the fluidity of the resulting composition to the point that the resulting composition is non-running at room temperature. A non-running composition is one that, if placed on a slight incline, will not experience substantial flow down the incline.

In another embodiment, the composition includes enough thickening agent to increase the fluidity of the resulting composition to the point that the resulting composition is non-running at body temperature. In yet another embodiment, enough thickening the composition includes enough thickening agent to increase the fluidity of the resulting composition such that the composition is substantially putty-like. In this embodiment, the composition will retain its shape even under mild pressure, such as that exerted by picking up a portion of the composition. This embodiment allows the composition to be molded to a shape such that it may be used in a predetermined location.

The thickening agent may be a polymeric compound. In one example, the thickening agent includes polyglycerylmethacrylate, as disclosed in U.S. Pat. No. 6,174,535 issued to Lundmark, incorporated herein by reference. The polyglycerylmethacrylate may be in the form of a transparent gel containing from about 50 to about 75% by weight solids, and may or may not contain incidental ingredients, such as propylene glycol which may be present in an amount of 2% or less. One example of a polyglycerylmethacrylate is Lubrajel CG, a registered trademark of United Guardian Inc., which is distributed by International Specialty Products of Wayne N. J. When combined with honey, the resulting composition may be described as a substantially homogeneous hydrated honey gel polymeric composition.

The composition may include other ingredients. The constituents of the composition of the present invention may be chosen so as to import certain attributes to the composition. For example, active pharmaceuticals, particularly to address concerns which extend beyond the ability of honey to address, or which augments or supplements the performance of honey may be included. Some examples of these active pharmaceuticals include fungicides, additional anti-bacterial agents and so forth.

The composition of the present invention may also include derivatives of the mangosteen plant. Derivatives of the mangosteen plant may include any composition that includes solids or liquids from a mangosteen plant. The derivatives of the mangosteen plant may be processed or unprocessed. Derivatives of the mangosteen plant may include extracts of the mangosteen plant, or any parts thereof, including the fruit thereof. In a non-limiting example, derivatives of the mangosteen plant may include the juice from the bark, leaves, or the fruit.

The Garcinia mangostana L. plant, or mangosteen plant, is known for a variety of uses in the areas to which it is indigenous. For example, there are a number of folk medicines in South-East Asia and Indonesia that employ various decoctions of the leaves, root, and bark of the mangosteen plant, as well as of the pericarp of the mangosteen fruit. For example, according to the literature, the thick mangosteen pericarp can be used as an astringent or in various decoctions for treating catarrh, cystitis, diarrhea, dysentery, eczema, fever, intestinal ailments, itch, and skin ailments. Other medicinal uses of the leaves, root and bark would be known to one of skill in the art. Also, the outer pericarp of the mangosteen fruit, which contains pectin, tannins, resins and a yellow latex, is used for treating and staining leather black.

In contrast to the thick outer pericarp, the edible inner pulp of the mangosteen fruit is widely regarded for its exquisite taste. The inner pulp of a single mangosteen fruit usually consists of four to eight juicy, white-colored segments. When preparing the white pulp segments for consumption, care must be taken so as to not stain the pulp segments with the resins and tannins and other matter that oozes out of the cut outer pericarp. The need to keep the delicious white pulp separate from the dark purple, staining, bitter pericarp has long been known to those familiar with the mangosteen fruit.

Though not intended to be limiting to the present invention, further benefits and uses of derivatives of the mangosteen plant include therapy for arthritis, specifically for pain relief. Mangosteen has been credited with several health benefits such as cancer prevention, anti-diarrhea, as an astringent or in various decoctions for treating catarrh, cystitis, diarrhea, dysentery, eczema, fever, intestinal ailments, itch, and skin ailments.

The composition of the present invention includes a therapeutic amount of derivatives of the mangosteen plant. The composition of the present invention includes at least about 1 weight percent, or at least about 3 weight percent, or at least about 5 weight percent; and less than about 25 weight percent, or less than about 15 weight percent, or less than about 10 weight percent derivative of the mangosteen plant.

Other optional ingredients may include: filler materials such as calcium carbonate, zinc oxide, barium sulfate and so forth, flexible or soft components, such as small beads; non-particulate materials, such as fibers; colors; alcohols; fragrances; other plant derivatives such as witch hazel, sage, piper, clove, ginger, red pepper, willow, rhubarb, sesame, chamomile, propolis, thyme, lavender, and so forth; plant oils such as cinnamon oil, flower or blossom oils, olive oil, palm oil, coconut oil, and so forth; beeswax; mineral oils; petrolatum; white petrolatum; phenylephrine HCl; shark liver oil; glycerin; pramoxine HCl; and so forth.

In one particular embodiment, the composition of the present invention includes only aloe, honey, and derivatives of the mangosteen plant. This embodiment may be particularly helpful for those who have an aversion to using a composition with an excess of additives. In this embodiment, the composition may be substantially devoid of added colors, corticoids, and fragrances, other than those present in the aloe, honey, and derivatives of the mangosteen plant. For example, one may have an allergy to certain colors, fragrances, or other common constituents of ointments. One with such an allergy could use the composition of the present embodiment because of the lack of these components. Yet others may have psychological, religious, sociological, or other aversions to using products with components that are not available without extensive processing. Thus, a composition with only honey, aloe, derivatives of the mangosteen plant, and thickener could be used by one with such an aversion.

In another similar embodiment, the composition may include only honey, aloe, a derivative of the mangosteen plant, and a thickening agent. As with the previous embodiment, this embodiment may be substantially devoid of corticoids, colors, and fragrances, other than those found in the honey, aloe, derivative of the mangosteen plant, and thickening agent. In one particular embodiment, the thickening agent includes only natural ingredients.

In another embodiment, the composition is includes a carrier. The carrier may be any used for the application of a topical composition to skin. Some non-limiting examples of carriers include: a suppository; ointment; cream; gel; paste; colloidion; glycerogelatin; liniment; lotion; paste; plaster; powder; tape; patch; aerosol; solution; and tincture.

The individual components of the composition may be mixed using any method known in the art to form the composition. In one embodiment, all of the individual constituents are added and mixed. In another embodiment, certain of the individual constituents are first mixed, and then the remaining individual constituents are mixed. In one embodiment, solid individual constituents are mixed with the honey and the aloe before the addition of any thickening agents. After the composition is thoroughly mixed, the thickening agent is added to the composition.

In one embodiment, the composition includes the following ingredients in their respective amounts: honey 70%; aloe 10%; bees wax 5%; propolis 5%; olive oil 5%; and mangosteen 5%. In another embodiment, the composition includes only honey (82%), aloe (12%), and mangosteen (6%).

The present invention also discloses a method of treating skin. The method includes applying a composition to the skin. The composition includes the composition of any embodiment illustrated above. The application of the ointment to the skin may be by any means known in the art. In one embodiment, the ointment is applied to the skin and allowed to dry. In another embodiment, the ointment is applied to the skin, and thereafter an article is placed over the ointment and the skin. In yet another embodiment, the ointment is applied to an article which is in turn applied to the skin.

The article of the latter embodiment may be any article capable of supporting the ointment and being applied to the skin. For example, the article may be an article that is intended to be applied to the skin. In another example, the article may be any article designed to be worn on the part of the body where the ointment is to be applied. The article may be designed to support the ointment or be worn on the part of the body where the ointment is to be applied. A combination of an article that supports ointment and an article designed to be worn on the part of the body where the ointment is to be applied may be used.

Some articles that are designed to support ointment include bandages, adhesive bandages, gauze pads, and so forth. Some articles that are designed to be worn on a part of the body where the ointment is to be applied include sleeves, socks, gloves, stretchable articles, hats, and so forth.

The area to be treated may be any area of irritated skin. Some non-limiting examples of irritation include: burns such as sunburns, chemical burns, rubbing burns, radiation burns and so forth; abrasions; cuts; episiotomies; sores such as irritations, allergic reactions, sore breasts, jock itch, cold sores, hemorrhoids, problems of portal circulation (hemorrhoids, congestion of the portal vein, inclination to venous thromboses), anal fistulas, inflammation of the anus, arteriosklerosis, purulent wounds and injuries, traumatic erysipelas, duodenal ulcer, migrating cheilitis, loss of hair, dandruff, alopecia, psoriasis, seborrheic dermatitis, athletes foot, external viral, fungal, or bacterial infections, eczema, rashes, ulcers, varicose veins, dermatomycoses, acne, pustules, burns, recovery from skin grafts, exudating wounds, chapped skin, hickeys, herpes sores, bruises, and so forth; insect bites; abrasions from plants such as poison ivy and so forth; animal bites; puncture wounds; and so forth.

Further disclosed, is the method of treating internal maladies using the compositions. In one embodiment, arthritis is treated by application of the composition to the skin surrounding the area affected by arthritis. The method of application may be any as disclosed above. In another embodiment, swelling is treated by application of the composition to the skin surrounding the area affected by the swelling. The method of application may be any as disclosed above. Further internal maladies that may be thus treated using the composition include, for example: joint pain; sprained joints; damaged tendons; damaged ligaments; damaged muscles; bruises; and so forth.

In one embodiment, the area of skin to be treated includes a hemorrhoid. The ointment is applied to an article designed to be worn and to support the ointment in the area of the hemorrhoid.

Also disclosed in this invention is a device for topical treatment. The device may include the composition of any of the above embodiments. The device also includes a substrate. The substrate may be any of the articles designed to support the composition or be worn on a part of the body where the ointment is to be applied disclosed above.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

For example, although the above description illustrates only a few articles able to support the composition, any article capable of supporting the composition is envisioned. For example, any piece of clothing may be used. Any substantially flat and flexible article may be used. More examples include a leaf, leather, paper, plastic, rubber, and so forth.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

In order to demonstrate the practice of the present invention, the following examples have been prepared. The examples should not, however, be viewed as limiting the scope of the invention. The claims will serve to define the invention.

1. A composition for application to irritated skin, comprising honey, aloe, and a derivative of mangosteen plant.

2. The composition of claim 1, wherein the honey is creamed.

3. The composition of claim 1, further comprising a thickening agent.

4. The composition of claim 3, wherein the thickening agent comprises an alginate based material.

5. The composition of claim 3, wherein the thickening agent comprises a polyvalent cation.

6. The composition of claim 1, wherein the aloe is powdered.

7. The composition of claim 1, wherein the honey comprises from about 50 weight percent, to about 75 weight percent, the aloe comprises from about 5 weight percent to about 20 weight percent, and the derivative of the mangosteen plant comprises from about 1 weight percent to about 10 weight percent.

8. The composition of claim 7, wherein the honey comprises about 70 weight percent, the aloe comprises about 10 weight percent, and the derivative of the mangosteen plant comprises about 5 weight percent, and further comprising about 5 weight percent bees wax, about 5 weight percent propolis, and about 5 weight percent olive oil.

9. A method of treating irritated skin, comprising:
applying a composition to irritated skin, said composition including honey, aloe, and a derivative of a mangosteen plant.

10. The method of claim 9, wherein the honey comprises creamed.

11. The method of claim 9, wherein the aloe is substantially devoid of water.

12. The method of claim 9, further comprising a thickening agent.

13. The method of claim 12, wherein the thickening agent comprises an alginate based material.

14. The method of claim 9, wherein the thickening agent comprises a polyvalent cation.

15. The method of claim 9, further including the step of applying the composition to a device.

16. The method of claim 9, wherein the irritated skin comprises one of the group consisting of: sunburns, chemical burns, rubbing burns, radiation burns, abrasions, cuts, episiotomies, irritations, allergic reactions, sore breasts, jock itch, cold sores, hemorrhoids, problems of portal circulation, anal fistulas, inflammation of the anus, arteriosklerosis, purulent wounds and injuries, traumatic crysipelas, duodenal ulcer, migrating cheilitis, loss of hair, dandruff, alopecia, psoriasis, seborrheic dermatitis, athletes foot, external viral, fungal, or bacterial infections, eczema, rashes, ulcers, varicose veins, dermatomycoses, acne, pustules, burns, recovery from skin grafts, exudating wounds, chapped skin, hickeys, herpes sores, bruises, insect bites, abrasions from poison ivy, animal bites, puncture wounds and combinations thereof.

17. The method of claim 9, wherein the honey comprises from about 50 weight percent, to about 75 weight percent, the aloe comprises from about 5 weight percent to about 20 weight percent, and the derivative of the mangosteen plant comprises from about 1 weight percent to about 10 weight percent.

18. The method of claim 9, wherein the honey comprises about 70 weight percent, the aloe comprises about 10 weight percent, and the derivative of the mangosteen plant comprises about 5 weight percent, and the composition further comprises about 5 weight percent bees wax, about 5 weight percent propolis, and about 5 weight percent olive oil.

19. A composition for application to hemorrhoids comprising, honey, aloe, and a derivative of a mangosteen plant.

20. (canceled)
